(19) United States
(12) Patent Application Publication
(10) Pub. No.: US 2009/0062793 A1
(43) Pub. Date: Mar. 5, 2009

(54) CUTTING WIRE ELECTRODE

(75) Inventors: Eliahu Eliachar, Haifa (IL); Nir Lilach, Kfar Yehoshua (IL); Dan Sade Hochstadter, Kibbutz Bet Alfa (IL); Gideon Meyer-Brodnitz, Haifa (IL)

Correspondence Address: Fleit Gibbons Gutman Bongini & Bianco PL, 21355 EAST DIXIE HIGHWAY, SUITE 115, MIAMI, FL, 33180, US

(73) Assignee: ROEI MEDICAL TECHNOLOGIES LTD., Katzrin (IL)

(21) Appl. No.: 12/162,944

(22) PCT Filed: Jan. 31, 2007
(86) PCT No.: PCT/IL07/00117

§ 371 Date: Aug. 13, 2008

Related U.S. Application Data
(60) Provisional application No. 60/763,387, filed on Jan. 31, 2006.

(51) Int. Cl. A61B 18/14

(52) U.S. Cl. 606/45; 606/49

(57) ABSTRACT

The present invention pertains to a cutting wire electrode, comprising at least one elongated shaft with a main longitudinal axis having at least one bore located at its distal portion, said bore being approximately perpendicular to said main longitudinal axis; and at least one wire loop located in the distal portion of said shaft; said loop comprising at least one continuous portion adapted to cut biological tissues, especially by electrocoagulation, and at least one abutment, wherein said abutment is adapted to be anchored within said bore and hence integrating said loop with said shaft.

### FIELD OF THE INVENTION

The present invention generally relates to cutting wire electrodes, and method of its production.

### BACKGROUND OF THE INVENTION

Most resectoscopes in use perform the resection with linear motion, whereby the forward and backward motion of a high electrical tension loop performs the surgical cut. Other resectoscopes are currently available which perform a similar function by using rotational motion, namely by Roei Medical Devices Ltd (IL) (‘Roei’), whereby the twisting motion of the high electrical tension loop performs the cut. Roei's technology is presented also in U.S. Pat. No. 6,971,989 ‘RESECTOSCOPE’; Patent application WO2005079682 ‘MEDICAL CUTTING TOOL WITH ADJUSTABLE ROTATING BLADE’; and Patent application WO03096912 ‘A WORKING TOOL FOR ACCURATE LATERAL RESECTION OF BIOLOGICAL TISSUE AND A METHOD FOR USE THEREOF’. In both systems, namely linear loop movement and the aforesaid Roei's rotational loop movement, the surgeon controls the motion of the distal loop edge (within the body cavity) from a handset attached to the shaft of the resectoscope located in the proximal (out of body) side.

Reference is made now to FIGS. 1A-C, presenting a side view of Roei's commercially available resectoscope (10), comprising an electrical loop (1) in its distal portion, said loop protruding from the very distal end of the rod (2), both being accommodated in a sheath (11) before use. A linear motion (3) of the handset provides for rotation (4) of loop (1) around the main longitudinal axis of the sheath (11). FIG. 1B presents a face view of the same. FIG. 1C illustrates a lateral cross section of a cutting wire electrode (20) disclosed in the art, characterized by rod (2), which comprises of two parallel bores being oriented perpendicular to said main longitudinal axis. Both ‘legs’ of the loop (1) are welded to said rod in two locations: distal location (5A) and proximal location (5B). Said configuration of the cutting wire electrode (1) on rod (2) as presented in prior art is that said rod is characterized by an abutment (21), a free end of rod (2) not being covered by wire (1), which is hindering, disturbing and precluding the ability to abut the cutting wire to the tissue for purposes of coagulating bleeding vessels. Moreover, said wire structure (10) is unstable for perpendicular physical forces.

Still in the prior art, reference is made to FIG. 1D, presenting a lateral cross section of cutting wire electrode (30), wherein the proximal portion of wire (1) is rolled up inside the rod (2), and is approximately parallel to the rod's main longitudinal axis (See distal leg 5A), while its proximal leg is inserted into a perpendicular bore in rod (2) (See proximal leg 5B). For example, in a non-limiting manner, the diameter of wire (1) is about 0.35 mm, and rod (2) diameter is about 1.10 mm. One drawback of cutting wire electrode (30) is its instability for radial forces, especially in its very distal portion. Another drawback is that welding of the leg (5a) is provided in arrangement 30 in the side of the resection, so that undesired oxidation of the tungsten-containing metal and peeling is obtained.

### SUMMARY OF THE INVENTION

It is one object of the present invention to disclose a cutting wire electrode. The electrode comprises of at least one elongated shaft with a main longitudinal axis having at least one bore located at its distal portion, the bore being approximately perpendicular to the main longitudinal axis; and, at least one wire loop located in the distal portion of the shaft; the loop comprises at least one continuous portion adapted to cut biological tissues, especially by electrocoagulation, and at least one abutment which is adapted to be anchored within the bore, hence integrating the loop with the shaft.

It is in the scope of the invention wherein this cutting wire electrode comprises of a first abutment and at least one second abutment; the abutment being anchored within the elongated shaft at the first bore and in the same bore or at least one second bore.

It is also in the scope of the invention wherein at least one wire loop is made of metal or metal alloy especially tungsten, wherein the cutting wire electrode is about 0.35 mm in diameter, and/or wherein the bore is perpendicular to the main longitudinal axis.

It is also in the scope of the invention wherein the cutting wire electrode with a plurality of bores; where at least one bore is not perpendicular to the main longitudinal axis, i.e., tilted bore.

It is also in the scope of the invention wherein at least one bore is parallel to the main longitudinal axis and located in its very distal end.

It is also in the scope of the invention wherein the distal abutment of the wire loop is anchored within the bore perpendicular to the longitudinal axis of the elongated shaft at the very distal end of the elongated shaft.

It is also in the scope of the invention wherein the distal abutment of the wire loop is anchored within the bore perpendicular to the longitudinal axis at some distance from the very distal end of the elongated shaft.

It is also in the scope of the invention wherein the wire loop is polygonal.

It is also in the scope of the invention wherein the proximal and distal abutments of the wire loop (50) are anchored within bores perpendicular to the longitudinal axis; and wherein the wire loop curves in the direction of the distal end of the elongated shaft.

It is also in the scope of the invention wherein the proximal and distal abutments of the wire loop are anchored within bores being perpendicular to the longitudinal axis of the elongated shaft; and wherein the wire loop curves in the direction of the proximal end of the elongated shaft.

It is also in the scope of the invention wherein the proximal abutment of the wire loop is inserted into a bore perpendicular to the elongated shaft and the distal abutment of the wire loop is inserted into a bore at the distal end of the elongated shaft such that the abutment lies approximately parallel to the longitudinal axis of the elongated shaft.

It is also in the scope of the invention wherein the proximal and distal abutments of the wire loop are anchored in bores being perpendicular to the longitudinal axis of the elongated shaft and in which the wire loop extends around the very distal end of the elongated shaft.

It is also in the scope of the invention wherein the wire loop abuts two elongated shafts, abutting the elongated shafts near the distal ends of the elongated shafts and anchored in bores perpendicular to the longitudinal axis of the elongated shafts.

It is also in the scope of the invention wherein the wire loop abuts two elongated shafts, abutting the elongated shafts at their very distal ends and anchored in bores approximately parallel to the longitudinal axis of the elongated shaft.

It is also in the scope of the invention wherein the wire loop abuts the elongated shaft and is anchored in a bore either (a) approximately parallel to the longitudinal axis of the elongated shaft and at its very distal end or (b) at angle of 0 to 90° to the longitudinal axis of the elongated shaft.

It is also in the scope of the invention wherein the wire loop circumscribes a void.

It is also in the scope of the invention wherein the length and dimensions of the wire loop are varied.

It is another object of the present invention to disclose a method for resecting biological tissues; comprising obtaining (i) a cutting wire electrode, comprising at least one elongated shaft with a main longitudinal axis having at least one bore located at its distal portion, the bore being approximately perpendicular to the main longitudinal axis; and, at least one wire loop located in the distal portion of the shaft; the loop comprising at least one continuous portion adapted to cut biological tissues, especially by electrocoagulation, and at least one abutment, wherein the abutment is adapted to be anchored within the bore, hence integrating the loop with the shaft; and, (ii) electrically wiring the shaft and the loop and hence, electrocoagulating the biological tissue.

It is also in the scope of the invention wherein the cutting wire electrode is placed at the site of the biological tissue to be removed.

It is also in the scope of the invention wherein the resecting step comprises rotation of the cutting wire up to 360° around the elongated shaft.

It is also in the scope of the invention wherein the resecting step comprises application of heat to the biological tissue either before or after the rotation and resection step.

It is also in the scope of the invention wherein the resecting step comprises application of vibration to the biological tissue.

It is also in the scope of the invention wherein application of heat cauterizes bleeding tissue by electrocoagulation.

It is also in the scope of the invention wherein the shape circumscribed by the void created by the cutting wire electrode controls the depth, shape and penetration of the cutting wire electrode.

It is another object of the present invention to disclose a method of producing a cutting wire electrode, comprising (i) obtaining at least one elongated shaft with a main longitudinal axis having at least one bore located at its distal portion, the bore being approximately perpendicular to the main longitudinal axis; and, (ii) obtaining at least one wire loop located in the distal portion of the shaft; the loop comprising at least one continuous portion adapted to cut biological tissues, especially by electrocoagulation, and at least one abutment, anchoring the abutment within the bore and hence integrating the loop with the shaft.

### BRIEF DESCRIPTION OF THE FIGURES

The objects and advantages of various embodiments of the invention will become apparent from the following description when read in conjunction with the accompanying drawings wherein

FIGS. 1a-1d schematically represent a side view of Roei's resectoscope with the high tension loop retracted into the outer sheath (prior art 1A), front view of the same (prior art 1B) and a lateral cross section of loop (1) in connection with rod (2) (prior art IC); FIG. 1D schematically represents a lateral cross section of cutting wire electrode (30) as presented in the prior art;

FIG. 2 schematically represents a lateral cross section of cutting wire electrode (40) according to another embodiment of the present invention;

FIG. 3 schematically represents a lateral cross section of cutting wire electrode (50) according to another embodiment of the present invention;

FIG. 4 schematically represents a lateral cross section of cutting wire electrode (60) according to another embodiment of the present invention;

FIG. 5 schematically represents a lateral cross section of cutting wire electrode (70) according to another embodiment of the present invention; and,

FIG. 6 schematically represents in a lateral cross section of cutting wire electrode (80) according to another embodiment of the present invention.

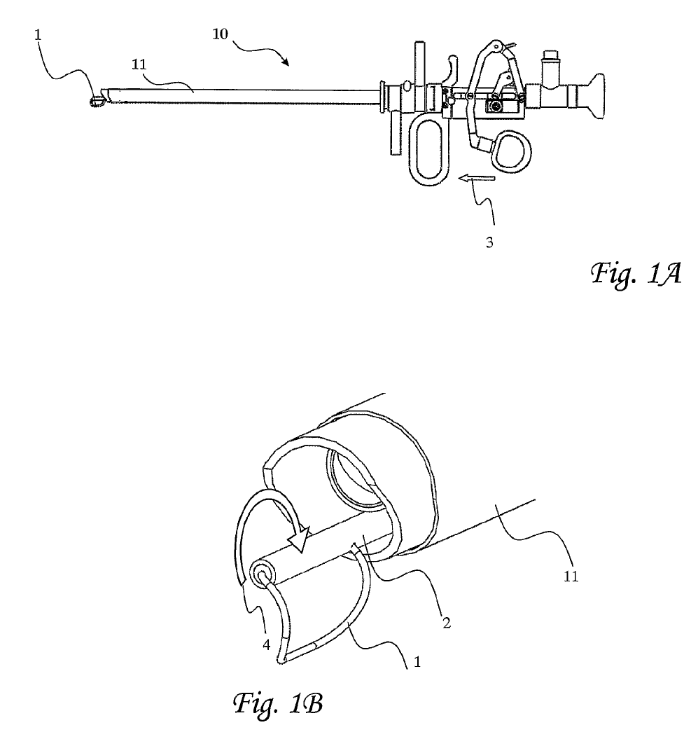

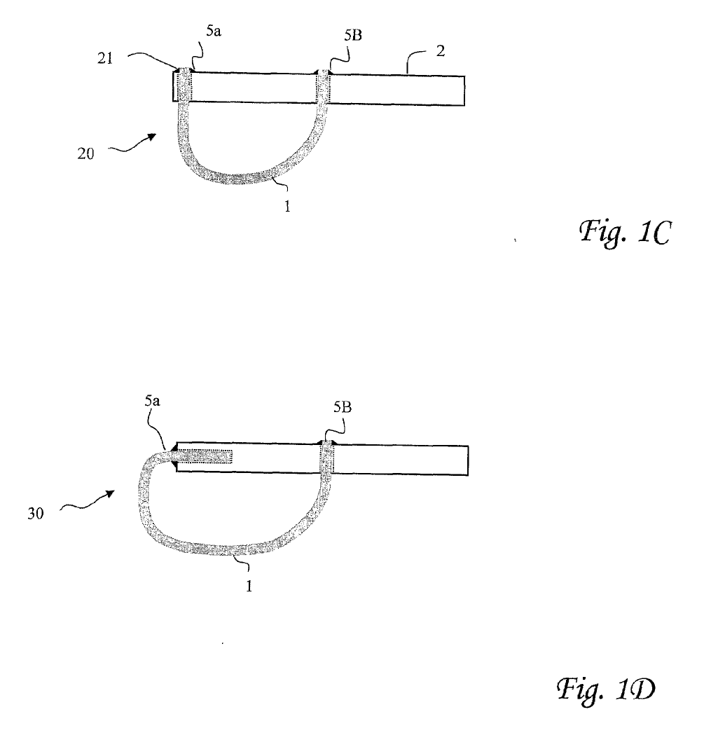

### DETAILED DESCRIPTION OF THE EMBODIMENTS

The following description is provided, alongside all chapters of the present invention, so as to enable any person skilled in the art to make use of said invention and sets forth the best modes contemplated by the inventor of carrying out this invention. Various modifications, however, will remain apparent to those skilled in the art, since the generic principles of the present invention have been defined specifically to provide a cutting wire electrode and a method of its production and use.

The term cutting wire electrode refers hereinafter to resectoscope wire, loop or any other resecting means. The cutting wire electrode is made in a non-limiting manner from metal or metal alloy. For example, a tungsten-containing metal is used. Said cutting wire electrode is connected to rod (2), made of any suitable metal-ware, e.g., stainless steel alloy. The shape, size and diameter of said cutting wire electrode can be widely utilized, and mainly depend on its surgical use.

The term resectoscope refers hereinafter especially to resectoscope but also to any endoscope, laparoscope or other electrically activated cutting wire electrode.

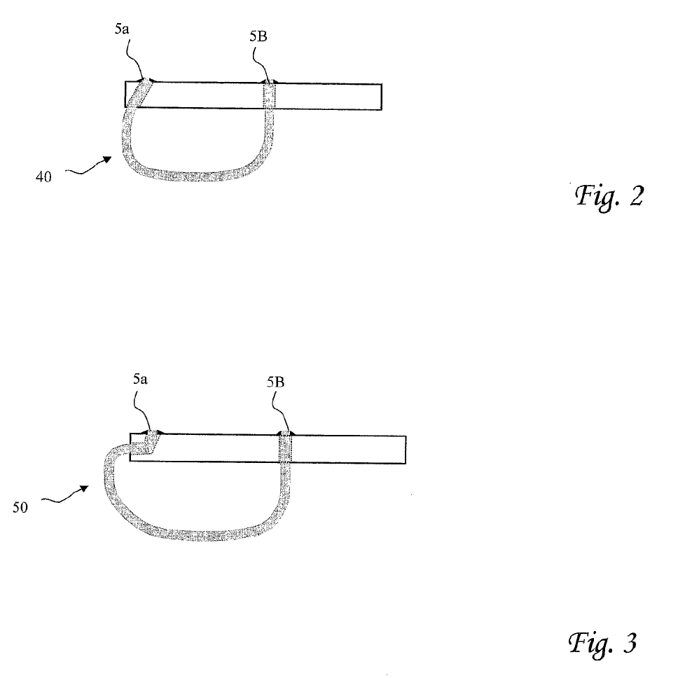

Reference is made now to FIG. 2, presenting a lateral cross section of cutting wire electrode (40) according to yet another embodiment of the present invention. Here, the proximal portion of the wire is located in front of the rod's abutment, and distal leg (5a) is inserted into the rod in a predetermined angle α, e.g., 0<α<90, so as the welding of the leg is provided in the rear side of the wire.

Reference is made now to FIG. 3, presenting a lateral cross section of cutting wire electrode (50) according to yet another embodiment of the present invention. Here, the proximal portion of the wire is located in front of the rod's abutment, and distal leg (5a) is inserted into the rod in parallel to the main longitudinal axis and then pushed there through to protrude the rod, leaving the rod in an angle α, so as the welding of said leg is provided in the rear side of the wire.

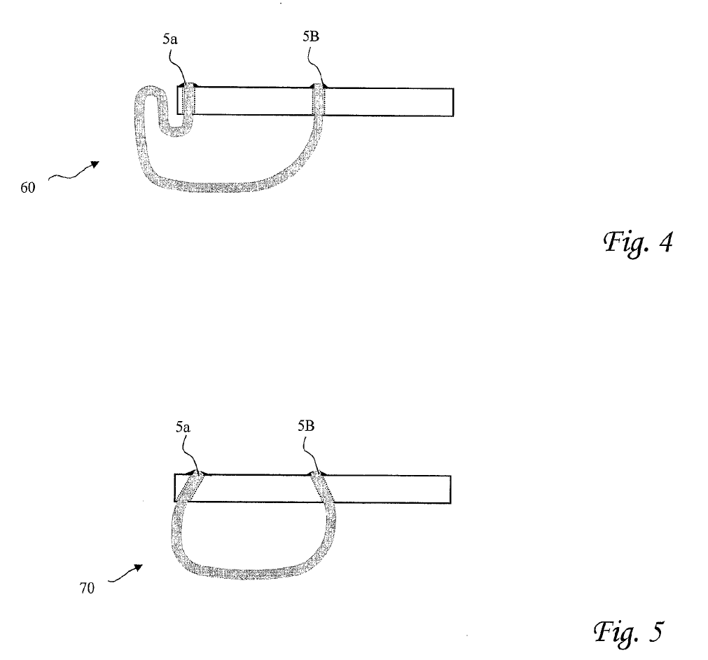

Reference is made now to FIG. 4, presenting a lateral cross section of cutting wire electrode (60) according to yet another embodiment of the present invention. Here, the proximal portion of the wire is located in front of the rod's abutment, and distal leg (5a) is inserted into the rod in an angle, so as the welding of said leg is provided in the rear side of the wire. Leg (5a) is inserted in the rod either in a perpendicular manner or in a given angle.

Reference is made now to FIG. 5, presenting a lateral cross section of cutting wire electrode (70) according to another embodiment of the present invention. Here, the proximal portion of the wire is located in front of the rod's abutment. Distal leg (5a) is inserted into the rod in a given angle, e.g., 0<α<90, and proximal leg is inserted into the rod in another given angle, e.g., 90<α<180, so as the welding of said leg is provided in the rear side of the wire.

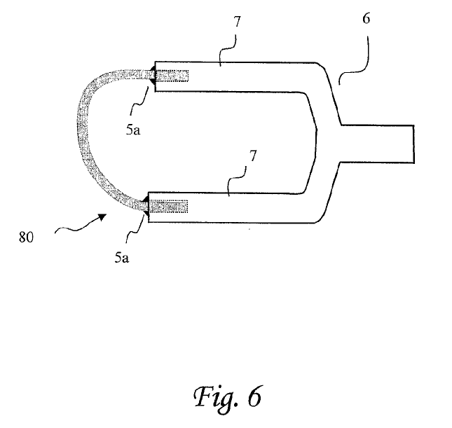

Reference is lastly made to FIG. 6, presenting a lateral cross section of cutting wire electrode (80) according to another embodiment of the present invention. Here each leg of the wire (5a) abuts each fork (7) of the rod (6) at the very distal end of each fork and is inserted into the front of each fork, perpendicular to the main axis of the fork.

The present invention also discloses a method of producing the cutting wire electrodes as defined and described in a non-exclusive manner above. The method comprises steps selected inter alia obtaining rod (2), providing a plurality of bores inside said rod, e.g., a distal bore and a proximal bore, inserting a wire (1) through said bores, so that at least one leg is partially located opposite to said wire. Optionally, said legs are welded to said rod to secure the wire's strength.

It is in the scope of the present invention, wherein at least one leg is immobilized to the rod by forcing the wire and/or rod, by means of an abrupt and intense force, e.g., a pulsed magnetic force or a force generated as a result of the discharge of electric current through a fluid as defined in U.S. Pat. No. 6,708,542 or in its literature.

Alternatively or additionally, it is in the scope of the present invention wherein at least one leg is immobilized to the rod by a process comprising also forcing electroless plating of nickel onto surfaces such as copper or fused tungsten as defined in U.S. Pat. No. 5,147,692 or in its literature.

Alternatively or additionally, it is in the scope of the present invention wherein at least one leg is immobilized to the rod by a process comprising also nickel plating using electrolytically refined nickel as the anode as defined in GB patent 991,634 or in its literature.

Alternatively or additionally, it is in the scope of the present invention wherein at least one leg is immobilized to the rod by a process comprising gluing the leg to the rod, heating the same and then pushing it into a bore etc.

1. A cutting wire electrode, comprising
a. at least one elongated shaft with a main longitudinal axis having at least one bore located at its distal portion, said bore being approximately perpendicular to said main longitudinal axis; and,
b. at least one wire loop located in the distal portion of said shaft; said loop comprising at least one continuous portion adapted to cut biological tissues, especially by electrocoagulation, and at least one abutment, wherein said abutment is adapted to be anchored within said bore and hence integrating said loop with said shaft.

2. The cutting wire electrode according to claim 1, comprising a first abutment and at least one second abutment; said abutment being anchored within the elongated shaft at the first bore and in the same bore or at least one second bore.

3. The cutting wire electrode according to claim 1, wherein at least one wire loop is made of metal or metal alloy especially tungsten.

4. The cutting wire electrode according to claim 1, wherein said cutting wire electrode is about 0.35 mm in diameter.

5. The cutting wire electrode according to claim 1, wherein said bore is perpendicular to said main longitudinal axis.

6. The cutting wire electrode according to claim 1 with a plurality of bores; wherein at least one bore is not perpendicular to said main longitudinal axis, i.e., tilted bore.

7. The cutting wire electrode according to claim 1, wherein at least one bore is parallel to said main longitudinal axis and located in its very distal end.

8. The cutting wire electrode according to claim 1, wherein the distal abutment of said wire loop is anchored within said bore perpendicular to the longitudinal axis of said elongated shaft at the very distal end of said elongated shaft.

9. The cutting wire electrode according to claim 1, wherein the distal abutment of said wire loop is anchored within said bore perpendicular to the said longitudinal axis at some distance from the very distal end of the said elongated shaft.

10. The cutting wire electrode according to claim 1, wherein the said wire loop is polygonal.

11. The cutting wire electrode (50) according to claim 1, wherein the proximal and distal abutments of said wire loop are anchored within bores being perpendicular to the said longitudinal axis; and wherein said wire loop curves in the direction of the distal end of the said elongated shaft.

12. The cutting wire electrode according to claim 1, wherein the proximal and distal abutments of said wire loop are anchored within bores being perpendicular to the longitudinal axis of said elongated shaft; and wherein said wire loop curves in the direction of the proximal end of the said elongated shaft.

13. The cutting wire electrode according to claim 1, wherein the proximal abutment of the wire loop is inserted into a bore perpendicular to the said elongated shaft and the distal abutment of the wire loop is inserted into a bore at the distal end of said elongated shaft such that the abutment lies approximately parallel to the longitudinal axis of said elongated shaft.

14. The cutting wire electrode according to claim 1, wherein the proximal and distal abutments of the wire loop are anchored in bores being perpendicular to longitudinal axis of the said elongated shaft and in which the wire loop extends around the very distal end of said elongated shaft.

15. The cutting wire electrode according to claim 1, wherein the wire loop abuts two elongated shafts, abutting the said elongated shafts near the distal ends of said elongated shafts anchored in bores perpendicular to the longitudinal axis of the said elongated shafts.

16. The cutting wire electrode according to claim 1, wherein the wire loop abuts two elongated shafts (80), abutting the said elongated shafts at their very distal ends anchored in bores approximately parallel to the longitudinal axis of the said elongated shaft.

17. The cutting wire electrode according to claim 1, wherein the wire loop abuts the said elongated shaft and is anchored in a bore either (i) approximately parallel to the longitudinal axis of the said elongated shaft and at its very distal end or (ii) at angle of 0 to 90° to the longitudinal axis of the said elongated shaft.

18. The cutting wire electrode according to claim 1, wherein the wire loop circumscribes a void.

19. The cutting wire electrode according to claim 1, wherein the length and dimensions of the wire loop are varied.

20. A method for resecting biological tissues; comprising obtaining (i) a cutting wire electrode, comprising at least one elongated shaft with a main longitudinal axis having at least one bore located at its distal portion, said bore being approximately perpendicular to said main longitudinal axis; and, at least one wire loop located in the distal portion of said shaft; said loop comprising at least one continuous portion adapted to cut biological tissues, especially by electrocoagulation, and at least one abutment, wherein said abutment is adapted to be anchored within said bore and hence integrating said loop with said shaft; and, (ii) electrically wiring said shaft and said loop and hence, electrocoagulating said biological tissue

21. The method of resecting biological tissue according to claim 20, wherein said cutting wire electrode is placed at the site of the biological tissue to be removed.

22. The method of resecting biological tissue according to claim 20, wherein said resecting step comprising rotation of the cutting wire up to 360° around the said elongated shaft.

23. The method of resecting biological tissue according to claim 20, wherein said resecting step comprising application of heat to the biological tissue either before or after said rotation and resection step.

24. The method of resecting biological tissue according to claim 20, wherein said resecting step comprising application of vibration to the biological tissue.

25. The method of resecting biological tissue according to claim 23, wherein application of heat cauterizes bleeding tissue by electrocoagulation.

26. The method of resecting biological tissue according to claim 20, wherein the shape circumscribed by the void created by the cutting wire electrode controls the depth, shape and penetration of the cutting wire electrode.

27. A method of producing a cutting wire electrode, comprising (i) obtaining at least one elongated shaft with a main longitudinal axis having at least one bore located at its distal portion, said bore being approximately perpendicular to said main longitudinal axis; and, (ii) obtaining at least one wire loop located in the distal portion of said shaft; said loop comprising at least one continuous portion adapted to cut biological tissues, especially by electrocoagulation, and at least one abutment, anchoring said abutment within said bore and hence integrating said loop with said shaft.
